(19) United States
(12) Patent Application Publication
(10) Pub. No.: US 2002/0016319 A1
(43) Pub. Date: Feb. 7, 2002

(54) Combined adamantane derivative and adrenergic agonist for relief of chronic pain without adverse side effects

(76) Inventors: John W. Olney, Ladue, MO (US); Nuri B. Farber, University City, MO (US); Vesna Jevtovic-Todorovic, Olivette, MO (US)

Correspondence Address: Patrick D. Kelley, 11939 Manchester # 403, St. Louis, MO, 63131, US

(21) Appl. No.: 09/820,263

(22) Filed: Mar. 28, 2001

Related U.S. Application Data
(63) Continuation-in-part of application No. 09/536,888, filed on Mar. 28, 2000.
Application No. 09/536,888 is a continuation-in-part of application No. 09/030,688, filed on Feb. 25, 1998, now abandoned.

(51) Int. Cl. A61K 31/554; A61K 31/553; A61K 31/13

(52) U.S. Cl. 514/211.13; 514/634; 514/662

(57) ABSTRACT

A combination of two drugs, from two different and previously unrelated categories, provides effective and long-lasting relief from neuropathic pain. Both drugs can be taken orally, in a convenient, painless, non-invasive manner that does not require injections. One drug in this combination is an α2 adrenergic agonist, exemplified by clonidine. The other drug in the combination is an adamantane derivative which has NMDA antagonist activity, such as memantine. Tests described herein demonstrate that when memantine is administered together with an α2 adrenergic agonist such as clonidine, these drugs mutually potentiate one another's neuropathic pain-relieving action, and provide potent and sustained neuropathic pain relief, even when each agent is administered at a low dosage that is below its threshold for causing adverse side effects. These results indicate that combining these two classes of drugs can provide safe and effective relief of neuropathic pain and possibly other types of chronic and/or intractable pain, without serious adverse side effects.

### RELATED APPLICATIONS

This application is a continuation-in-part of patent application Ser. No. 09/536,888, filed on Mar. 28, 2000, which in turn was a continuation-in-part of application Ser. No. 09/030,688, filed on Feb. 25, 1998, now abandoned.

### GOVERNMENT SUPPORT

The research which led to this invention was supported in part by a grant from the National Institutes of Health. Accordingly, the federal government has certain rights in this invention.

### BACKGROUND OF THE INVENTION

The present invention relates to neurology and pharmacology, and to drugs which can treat and control various types of chronic pain (including neuropathic pain) without causing adverse side effects.

Currently available drug treatments for chronic and severe pain are subject to various limitations and shortcomings. Drugs such as ibuprofen, naproxen, acetaminophen, and aspirin do not have nearly the same potency as opiates (such as morphine) for treating severe pain, but opiates are excessively sedating; indeed, they are often referred to as narcotics, since they typically induce drowsiness and sleep, and interfere with mental clarity in ways that hinder a patient's ability to work, drive, or carry on normal and healthy family and personal activities. Opiates also have other adverse side effects (including gastrointestinal disorders, sexual dysfunctions, etc.), and they pose very high risks of tolerance, dependence, and addiction.

Another important shortcoming of opiates and similarly potent pain-relieving drugs is that they are not effective in treating certain types of pain, the most notable example being “neuropathic pain”, a type of pain experienced when the patient's nervous system is itself suffering from some type of pathological damage or condition (hence the term “neuro-pathic”).

There are several different types of neuropathic pain, but a common denominator of all types is that they do not respond adequately to opiate medications. Indeed, this trait is so prevalent that physicians tend to view “neuropathic pain” as being synonymous with pain that does not respond adequately to opiates. When a pain condition in a specific patient will not respond adequately to opiates, it is assumed that: (i) the patient is suffering from neuropathic pain; and, (ii) the neuronal pathways that are causing or aggravating the pain condition in that patient are not pathways that use opiate receptors; rather, they convey pain messages via other neurotransmitter/receptor systems.

Most cases of neuropathic pain appear to involve chronic conditions that arise when nerve fibers or endings in a certain part of the body (or the larger neuronal networks they are connected to, which may include neurons located in the spinal cord) have become hyper-sensitive (also referred to as being hyper-irritable, or being in a “kindled” or “wind-up” condition). In this condition, certain neuronal endings, receptors, or other components or circuits are in a chronic state of abnormally high sensitivity, and/or have abnormally low triggering thresholds. In this state, they convey (either spontaneously, or in response to very mild stimuli that would not be painful to a healthy person) far too many nerve signals of the type classified by neurologists as “nociceptive” nerve impulses (i.e., nerve impulses interpreted by the nervous system as signalling pain; this class of signals is distinct from other types of nerve impulses, such as sensory signals for light, smell, taste, etc.).

A person with a hypersensitized or “kindled” neuropathic condition is comparable to a person whose eyes were dilated by a drug, during an eye exam, and who is then forced to look directly into a bright light. Just as bright light is very unpleasant and even painful to a person with dilated eyes, a patient subject to neuropathic pain will suffer from what appear to be artificially amplified surges and waves of nociceptive nerve signals, from the affected part(s) of the body. Those amplified surges and waves will be perceived as pain, even when no stimulus has been inflicted that would be perceived as painful by a person who is not suffering from the neuropathic hypersensitivity.

Well-known examples of neuropathic pain include: (i) chronic painful states that occur in association with diabetes, often referred to as “diabetic neuropathy”; (ii) chronic pain associated with traumatic injury to the peripheral nervous system; (iii) chronic pain resulting from herpes zoster (also known as shingles, or post-herpetic neuropathy) or similar infections that attack and damage nerve fibers or endings; (iv) post-operative pain, which arises after surgery and then lingers far beyond a normal convalescent period; (v) “phantom limb” pain, in which an amputee suffers from feelings of pain or discomfort that seems to originate in the missing limb; and (vi) causalgia, which refers to pain that is perceived as a burning sensation (“causalgia” comes from the same Greek root as “cautery”, and has nothing to do with causation).

In addition, certain patterns of neuropathic pain commonly arise in certain parts of the central or peripheral nervous system. Examples include trigeminal pain, which afflicts the trigeminal nerve in the facial region, and arachnoiditis, in which a certain layer of membrane that generally surrounds the brain and spinal cord becomes damaged and/or inflamed, usually due to trauma, surgery, or infection.

All of these conditions appear to share at least some underlying mechanisms and/or traits. In particular, they share the common distinction that they all involve chronic pain, and the pain reaches a level regarded by the patient and the treating physician as “intractable”. As used herein, the term intractable implies that: (i) a pain condition cannot be adequately relieved by opiates or other currently available pain medications; and (ii) any short-term relief provided by opiates or other drugs is usually accompanied by serious side effects, such as sedation, gastrointestinal disorders, etc.

Any references herein to relief, short-term relief, or similar terms, refer to an effective form of non-sedating relief. Obviously, if a patient is rendered unconscious by a powerful sedative, the patient will feel no pain for as long as he or she remains unconscious. However, just as obviously, that is not the goal of an effective treatment for chronic pain. In this context, terms such as sedative, sedating, or sedation do not imply a treatment that is soothing, relaxing, gentle, or otherwise benevolent and appreciated; instead, terms such as sedative or sedation imply a highly undesired and possibly even debilitating interference with mental clarity and acuity, and should be regarded in the same general category as “addictive narcotic”.

One of the worst and most troubling and depressing side effects of pain-killing drugs, in the view of anyone who must take them to treat chronic intractable pain, is the severe disruption they impose on a patient's mental clarity. Sedating pain-killers make it tiring and difficult, and often impossible, for a patient to have the sort of normal, healthy and happy personal, family, and work activities and relationships that the person enjoyed before the chronic pain began. For people suffering from chronic pain, sedation does not imply relaxing, or being able to get to sleep easily. Instead, sedating side effects imply lethargy, and a severe loss of energy, initiative, enthusiasm, and mental clarity. Sedation implies chronic difficulty in keeping a house neat, doing a good job at work, and difficulty in carrying on even a simple normal conversation. Sedation also substantially increases the risk of serious accidents, such as a fall while walking (which poses a serious risk of a broken hip, leg, or arm, especially in elderly patients), a collision while driving, etc.

If an effective and non-sedating treatment for neuropathic or other chronic or intractable pain could be discovered and made publicly available, it would be a major advance over opiates and other currently available drug treatments, and it would offer blessed relief to literally millions of people around the world.

This invention discloses what appears to be a major advance in that direction. To understand how this drug treatment works, and which drugs are covered by certain terms used in the text and claims below, background information needs to be provided on the NMDA subclass of glutamate receptor, and on so-called “NMDA antagonist” drugs.

NMDA Receptors and NMDA Antagonists

Glutamate receptors (one of the most important classes of excitatory neuronal receptrs in mammals) are described in various references, such as Principles of Neuroscience (E. R. Kandel, et al, editors, McGraw Hill, 4th ed., 2000), and in numerous review articles. One subclass of glutamate receptors is powerfully activated by a probe drug called N-methyl-D-aspartate (abbreviated as NMDA), so those receptors are called NMDA receptors. Inside the CNS, these receptors are triggered mainly by glutamate (the ionized form of glutamic acid) and to a much lesser extent by aspartate.

Because of certain physiological and cellular factors, glutamate can begin accumulating at abnormally high concentrations in the synapses between CNS neurons, if a major insult (such as a stroke, head injury, cardiac arrest, near-drowning, etc.) leads to a crisis inside the brain or spine which involves ischemia (inadequate blood flow) and/or hypoxia (inadequate oxygen supply). This process, which can severely aggravate brain or spinal damage following a crisis, is called “excitotoxicity”. By the late 1980's, it was becoming established that excessive activation of NMDA receptors can seriously aggravate and increase excitotoxic brain damage; published studies are reviewed in Olney 1990 and Choi 1992.

In response, a number of pharmaceutical companies began developing NMDA “antagonist” drugs (i.e., drugs which can suppress the normal triggering of NMDA receptor-mediated neuronal firings by the actions of glutamate at those receptors). By the mid-1980's, a number of NMDA antagonist compounds had become available to neurology researchers, who began testing these compounds to evaluate their effects on nearly every conceivable type of neurological function, including pain, sensory processing, memory, etc.

However, by the late 1980's, neurologists were also realizing that NMDA antagonist drugs cause serious side effects, including psychotic reactions and physical injury to neurons, when administered at the dosages required to relieve neuropathic pain, or to prevent excitotoxic neuronal damage after a stroke or other acute insult.

In humans, NMDA antagonist drugs cause psychotic side effects, including hallucinations and delusions. People who abuse phencyclidine (also known as PCP, or “angel dust”) often suffer acute psychotic episodes, and surgical patients emerging from ketamine anesthesia often suffer “emergence” psychoses unless they are also treated with a second drug to suppress that reaction. Both phencyclidine and ketamine are NMDA antagonists.

In animals, analysis of brain tissue following treatment with an NMDA antagonist drug reveals that NMDA antagonists cause acute physical injury in certain neurons. These neurotoxic effects occur in several specific known regions of the brain, including two regions known as the posterior cingulate and retrosplenial (PC/RS) cortex, as well as the hippocampus. These brain regions receive input from a variety of other brain regions, and they perform important “central processing” functions (which can also be regarded as “switchboard” or “clearinghouse” functions).

The damage that occurs in the most vulnerable portions of the brain is manifested, at a fairly early stage, by the formation of empty “vacuoles” inside the affected neurons. These vacuoles, which do not occur in healthy neurons, provide a useful and convenient way to measure and quantify the damage to vulnerable neurons, since the vacuoles can be easily seen and counted, using microscopic examination of tissue slices. Other types of neuronal damage can also be detected if the various analytical steps are taken; as one example, affected neurons begin expressing “heat shock” proteins, which indicate that the neurons containing those proteins are being subjected to severe and potentially lethal stress. In addition, potent NMDA antagonists at substantial dosages can cause neuronal death, which can be measured using various types of stains that cannot permeate into viable neurons. Neuronal vacuoles, heat shock protein expression, mitochondrial damage, and other neurotoxic manifestations are discussed in more detail in items such as U.S. Pat. No. 5,877,173 (Olney et al 1999).

There is considerable evidence indicating that the same regions of the brain which display the most damage from NMDA antagonist neurotoxicity, in tissue samples from test animals, are also involved in causing psychotic effects in humans. Accordingly, evidence that the same neural networks are involved in psychotic reactions in humans, and neurotoxic damage in animals, strongly suggests that humans who take NMDA antagonist drugs, in addition to suffering transient effects such as hallucinations, are at a serious risk of the same type of physical injury to neurons that can be shown to occur in test animals.

Because NMDA antagonist drugs are known to cause psychoto-mimetic effects in humans, and are strongly suspected of posing serious and unacceptable risks of permanent brain damage, efforts to develop NMDA antagonist drugs for neuroprotective purposes following a stroke or similar crisis, or for use in treating neuropathic pain or other chronic pain, have uniformly failed. Even though at least a dozen major drug companies launched major research programs, attempting to develop effective yet safe NMDA antagonists, every such effort has failed. As this is being written, in March 2001 (nearly 20 years after the first known NMDA antagonist drugs were announced), not a single known NMDA antagonist drug has ever been approved for public sale and use, by the U.S. Food and Drug Administration.

The only publicly available drugs that are known to have some degree of NMDA antagonist activity fall into two categories. The first category includes two surgical anesthetics, ketamine and tiletamine, and these were approved by the FDA many years before their role as NMDA antagonists were known. The second category includes a few drugs (such as dextromethorphan and procyclidine) which have strong primary activities involving completely different neuronal receptor systems, and which were discovered, after many years of public use, to have relatively mild and weak secondary activities as NMDA antagonists.

The failure of any drug company to successfully launch any known NMDA antagonist drug as a commercial product is also noteworthy, and perhaps even peculiar, because it has been known for nearly 10 years that any of several types of drugs can effectively block the neurotoxic side effects of NMDA antagonists, in test animals. When used for this purpose, these drugs are referred to herein as “safener” drugs, borrowing a term that is well known in other chemical industries, such as the herbicide industry. Administration of one or more of these “safener” drugs can effectively block the neurotoxic side effects caused by NMDA antagonists, in test animals.

These types of “safener” drugs can be divided into several categories, depending on which neurotransmitter system they affect. Briefly, they include: (1) anticholinergic drugs, such as scopolamine, as described in Olney et al 1991 and U.S. Pat. No. 5,034,400 (Olney 1991); (2) GABA agonist drugs, as described in U.S. Pat. No. 5,474,990 (Olney 1995); (3) alpha-2 (α2) adrenergic agonists, discussed in detail below; and, (4) drugs that suppress activity at kainate and AMPA receptors, as described in U.S. Pat. No. 5,767,130 (Olney 1998).

Despite all these options (and several others, not mentioned above), most of which involve well-known and long-accepted drugs, and despite clear demonstrations in test animals that the neurotoxic side effects of even the most potent NMDA antagonists can be greatly reduced or entirely eliminated by these types of “safener” drugs, the pharmaceutical industry has steered entirely away from any effort to develop and commercialize any drug combination involving a potent NMDA antagonist drug accompanied by a second drug that would prevent the neurotoxic side effects of the potent NMDA antagonist drug. Instead, efforts have continued to focus on a search for an NMDA antagonist drug that would be not cause neurotoxic side effects, at the dosages needed to exert a beneficial neuroprotective effect.

Because of the prominent role one of the Inventors herein (Olney) played, first in elucidating the neurotoxic effects caused by excessive quantities of glutamate (Olney 1969), then in clearly locating and identifying the types of measurable neuronal damage that were being inflicted by NMDA antagonist drugs in the brains of lab animals (Olney et al 1989), Olney's laboratories were frequently requested to evaluate the neurotoxic risks of new candidate drugs that were becoming the heavily-investigated “lead compounds” in a number of drug companies' efforts to find a safe NMDA antagonist.

However, in every such investigation, Olney's tests disclosed that, if an NMDA antagonist drug was used in a quantity sufficient to actually reduce excitotoxic brain damage following a stroke or other brain insult, it also caused neurotoxic vacuoles in the PC/RS regions of the brain. Olney's research repeatedly revealed that even weak NMDA antagonists (such as dextromethorphan, the well-known cough suppressant that has been used for decades in cough syrups) would begin causing neurotoxic effects, if administered at dosages sufficient to reduce excitotoxic brain damage following a hypoxic crisis.

In the final analysis, the problem was not that the NMDA antagonist drugs being tested were all too potent to be used safely; instead, the problem was in certain inherent and unavoidable traits of the neuronal circuitry inside the brain. Glutamate molecules and NMDA receptors are so heavily and deeply involved, in so many crucially important circuits and regions inside the brain, that if any NMDA blocker drug is administered at a dosage which is potent enough to substantially slow down the excitotoxic processes triggered by excess glutamate accumulation inside a brain region suffering from a hypoxic crisis such as a stroke or cardiac arrest, then that protective dosage of that NMDA blocker drug will also be potent enough to trigger major disruptions and derangements in other crucially important neuronal circuits, as well.

In other words, because of the ubiquity of the NMDA receptor system inside the brain, any drug which blocks NMDA receptors at levels that can help reduce excitotoxic cell death, after a brain crisis, will also trigger major disruptions and derangements in other neuronal circuits. These disruptions and derangements are of such a type and magnitude that they lead to toxic damage inside the brain, affecting neurons in the cerebral cortex which perform important “clearinghouse” or “switchboard” functions.

In view of those consistent and repeated results from dozens of different drug tests on animals, Olney concluded that the best way, and probably the only realistic way, to reduce and prevent the neurotoxic side effects of NMDA antagonists was not by developing better NMDA antagonists, but by instead developing ways of coadministering NMDA antagonists along with other classes of drugs. These secondary drugs are referred to herein as “safener” drugs, borrowing an old chemical term that has been used for many years in the herbicide industry.

Memantine is at the focal point of one effort to develop an inherently safe NMDA antagonist, which can be administered safely by itself, with no accompanying safener drug. Since memantine is highly important in this invention, it is discussed in greater detail under the next subheading.

Memantine and Other Adamantane Derivatives

Memantine is an analog of adamantane, a compound that occurs naturally in petroleum. Adamantane, which is also called tricyclo-decane, has a four-sided structure, comparable to a pyramid, wherein all four sides of the pyramid are formed by cyclohexane rings that share atoms with each other.

In the 1960s, researchers discovered that compounds in the adamantane family, especially an analog named amantadine, have some degree of anti-viral activity (U.S. Pat. Nos. 3,328,251, Smith 1967; 3,391,142, Mills et al 1968). Amantadine was subsequently found to have muscle relaxant properties, and it was marketed in the US as a treatment for parkinsonism. Amantadine is currently availaable in the U.S., both as a generic drug and under the trademark SYMMETREL (sold by Endo Laboratories, Wilmington, Del.).

Additional research on adamantane analogs revealed that 3,5-dimethyl-1-aminoadamantane (called memantine) has the same muscle relaxing effects as amantadine, and in some respects works better as a drug for treating patients with Parkinsonism. Accordingly, in the 1990s, memantine was approved for sale and public use in Europe, under the trademark AKATINOL. Based initially on anecdotal reports, it was also reported that memantine might be helpful in treating at least some types of dementia (e.g., Jain 2000).

About 10 years ago, it was reported that memantine has mild NMDA antagonist activity (Bormann 1989; Kornhuber et al 1994; also see U.S. Pat. No. 5,061,703, Bormann 1991)). This led to several patent filings, in an effort to claim the potential use of memantine and amantadine in reducing neuronal damage associated with progressive neurodegenerative diseases (U.S. Pat. No. 5,614,560, Lipton 1997) or AIDS dementia (U.S. Pat. No. 5,506,231, Lipton 1996), and in treating neuropathic pain (U.S. Pat. No. 5,334,618, Lipton 1994).

Neugebauer et al 1993, Eisenberg et al 1993, 1994, 1995, and Carlton et al 1995 also reported early research resuls on memantine for treating neuropathic pain; however, the accumulated reports on pain control are mixed at best, and several recent research reports (e.g., Eisenberg et al 1998, Nikolajsen et al 2000) stated that at the dosages tested, memantine failed to provide any significant pain relief in human patients who were tested. Von Euler et al 1997 and Tzschentke et al 1999 also provide negative reports on the apparent lack of efficacy of memantine in treating certain other conditions.

Nevertheless, other reports state that memantine is highly promising, and may be effective in treating a host of neurological disorders. Such reports include Chen et al 1998, Jain 2000, Zinkevich et al 2000; also see Parsons et al 1999, written by employees of the Merz company. These reports all indicate that memantine, at effective dosages, is inherently safe and essentially free from the neurotoxic side effects that have plagued other NMDA antagonist drugs, due to the fact that the NMDA receptor “kinetics” of memantine are different from those of drugs such as MK-801; apparently, memantine releases and disengages from the NMDA receptor complex much more quickly than neurotoxic NMDA antagonists such as MK-801. Therefore, at the present time, a concerted effort is being made by the manufacturers of memantine (Merz, a German pharmaceutical firm) to gain approval from the U.S. Food and Drug Administration (FDA) to market memantine in the US as a non-neurotoxic NMDA antagonist.

To support their claims that memantine is free from neurotoxic effects, even when administered at a dosage that can protect against excitotoxic brain damage, memantine proponents state that they have administered memantine to rats at dosages of 20 mg/kg, and reportedly did not find signs of neurotoxicity in the retrosplenial cortex (e.g., Chen et al 1998). To support their claims that memantine is an effective neuroprotective agent at that same dosage, they tested that same dose of memantine (20 mg/kg) and reported that it provided neuroprotection against ischemic degeneration of neurons in the adult rat brain (Chen et al 1998). Thus, memantine is being publicly represented as a non-neurotoxic NMDA antagonist that is effective in treating neurological disorders.

The Applicants herein evaluated whether memantine causes neurotoxic changes in the adult rat brain, when injected intraperitoneally at 25, 30, 50, 75 and 100 mg/kg. It was found in these tests that, at every one of those doses (including the 25 mg/kg dosage rate), memantine did indeed cause neurotoxic changes, including vacuole formation in neurons in the vulnerable PC/RS regions of the rat cerebral cortex. Although only a small number of neurons were injured at the 25 mg/kg dosage, neuronal injury was decidedly present and easily visible, even at the 25 mg/kg dosage. Progressively larger numbers of neurons were injured at each of the higher doses tested.

Therefore, while the proponents of memantine may be correct in claiming that at 20 mg/kg, memantine did not cause neurotoxic changes in rat brains, it is equally correct to say that beginning at 25 mg/kg, memantine begins to generate neurotoxic signs in adult rat brains, and the neurotoxic damage escalates from that point in a dose-dependent manner. It would also be correct to say that, in medical practice, there would be relatively little margin of safety between (i) the dose that would be required for a substantial therapeutic benefit, in reducing excitotoxic brain damage following a stroke or similar crisis, and (ii) the dose that would begin to pose serious risks of neurotoxic side effects. This risk is especially large in patients who have suffered strokes, head traumas, or similar cerebral crises in which blood flow through the brain is no longer uniform and consistent, comparable to a healthy brain.

The Applicants also evaluated the neuropathic pain-relieving properties of memantine, when injected intraperitoneally at 2.5, 5 and 10 mg/kg into adult rats. In these tests, it was found that memantine had no effect on neuropathic pain at the lower doses, but at 10 mg/kg, it provided a detectable level of relatively brief short-term relief. The Applicants did not evaluate any higher doses for treating neuropathic pain, because several crucial differences exist between a treatment that will be used chronically (e.g., taken every day, or even several times a day, for months or years), and a one-time acute treatment to try to minimize permanent brain damage following a stroke or other acute CNS crisis.

One factor can be summarized as follows. Various reports and the Applicants' own tests all supported the conclusion that a dosage of roughly 20 mg/kg was the maximum dose that could be tolerated safely, in a one-time dosage situation, in response to an acute CNS crisis. However, because of various factors (including drug half-lives, drug accumulation, and metabolite accumulation), the maximum dosage that can be tolerated safely, if a potentially neurotoxic compound is taken every day for months, is substantially lower that the maximum safe one-time dosage. The dangers in this type of situation are not comparable to the problems of tolerance and dependence, where a patient who keeps taking the same drug over and over begins to need more and more of the drug, in order to sustain the same level of effect; instead, the dangers are analogous to moving closer and closer to a threshold, where another dosage of the same compound might push a patient beyond that threshold, to a pont wherein some type of damage-related cascade will kick in, and begin inflicting large amounts of damage in response to the same small and incremental dosage that had not triggered any detectable damage previously.

This type of situation, involving accumulating stress that leads to serious damage due to repeated administration of low dosages of NMDA antagonists, has indeed been seen by the Applicants, enough times and with enough consistency to make it a valid generalization. As an example, if a relatively small quantity of MK-801 is administered to rats a single time, it will produce an entirely reversible vacuole reaction, which will disappear entirely and leave no trace within a week. However, if that same low dosage is administered to rats each day, for four days in a row, it will cause a serious severe damage.

Accordingly, based on their experience in working with a wide assortment of candidate NMDA antgaonist compounds, the Applicants herein concluded that the maximum safe dosage of an NMDA antagonist drug which could be taken chronically is likely to be only half (or less) of the maximum dosage that can be tolerated safely based on a single exposure to the drug. Since 20 mg/kg was reportedly the maximum safe dosage of memantine, when administered to an animal only one time, their assumption was that half of that dosage (i.e., 10 mg/kg) was the maximum dosage that would be worth testing, for possible use as a drug that might be taken, every day, to treat neuropathic pain, or other chronic pain.

The second factor, which indicated that 10 mg/kg of memantine was the maximum dosage that merited testing for possible use as a chronic treatment for neuropathic pain, was based on research done in Olney's labs on other NMDA antagonists, described in articles such as Wozniak et al 1990 and Jevtovic-Todorovic et al 1998. When an NMDA antagonist is being studied, a “neurotoxic threshold” dosage can be determined for that drug. This neurotoxic threshold is the dosage of a drug which, when administered a single time to an animal, causes vacuoles and other easily detectable damage to begin appearing, in clear and consistent form, in neurons in the PC/RS cortex and certain other vulnerable regions of the brain.

The neurotoxic threshold number is important, for an NMDA antagonist, because other research in Olney's labs, on a variety of candidate NMDA antagonist drugs, showed that substantial behavioral and/or cognitive impairments (including sedation, loss of motor skills, inability to perform adequately on water maze tests and similar tests of memory, etc.) begin to be clearly manifested at dosages which are roughly one-half of the “neurotoxic threshold” dosage, in any given NMDA antagonist drug.

Accordingly, since 25 mg/kg had been established as being at or close to the “neurotoxic threshold” for memantine, the dosage that was expected to begin causing sedation, loss of motor skills, and memory impairments was somewhere in the range of about 10 to about 15 mg/kg. In view of that threshold for impaired cognition and behavior, it was clear to the Applicants herein that if 10 mg/kg of memantine was required to obtain even brief relief from neuropathic pain, that dosage level was not safe for chronic use; it was too close to the dosage that would begin inducing memory impairments, loss of motor skills, etc. Accordingly, even if the 10 mg/kg level for a chronic daily dosage would not trigger frank neurotoxic changes (i.e ., lasting physical damage to the brain), it clearly could not and would not offer a useful treatment for neuropathic pain.

For these reasons, based on their direct testing of memantine and their prior experiences in studying numerous other candidate NMDA antagonist drugs, it was clear to the Applicants that memantine, by itself, could not offer a safe yet adequate and effective treatment which could be used, every day, to help control neuropathic pain.

Before they had received and tested memantine, the Applicants had already commenced a systematic program of evaluating different combinations of drugs, to determine whether some combination might offer improved relief from neuropathic pain. That search focused mainly on combinations of (i) NMDA antagonists, combined with (ii) other types of drugs which had been previously shown to function as “safener” drugs when coadministered with NMDA antagonists.

In this context, a “safener” drug is a drug which, if coadministered with an NMDA antagonist, can reduce or block the neurotoxic side effects (vacuoles, etc.) of the NMDA antagonist. The term “safener” was borrowed from the herbicide industry; it relates to compounds that can be sprayed on a field, several days before a certain herbicide is sprayed on the field. Through complex biochemical mechanisms, a good safener will cause the crops to become substantially more resistant to that particular herbicide. This allows a substantially higher load of herbicide to be sprayed on a safener-treated field.

Returning to NMDA antagonist drugs, a substantial number of such safener drugs had been identified by Olney et al, beginning in about 1990. By 1999, the list included (1) anticholinergic drugs, such as scopolamine, as described in Olney et al 1991 and U.S. Pat. No. 5,034,400 (Olney 1991); (2) GABA agonist drugs, as described in U.S. Pat. No. 5,474,990 (Olney 1995); (3) alpha-2 (α2) adrenergic agonists, discussed in detail below; and, (4) drugs that suppress activity at kainate and AMPA receptors, as described in U.S. Pat. No. 5,767,130 (Olney 1998).

Since α2 adrenergic agonists were known to be able to reduce the toxic side effects of NMDA antagonists, they were included as one of the categories of drugs that were included in the screening process. This was done, not as a logical step, but as part of “casting a wide net”. Even at that time, the Applicants knew that because of two factors discussed below, α2 adrenergic agonists were not likely to be useful, for daily treatment of a chronic condition such as neuropathic pain.

The Adrenergic System; α2 Agonist Drugs

The “adrenergic” system is discussed in textbooks such as Principles of Neuroscience (Kandel et al, editors, 2000), and the Encyclopedia of Neuroscience (first edition, edited by Adelman, 1987, or second edition, edited by Adelman and Smith, 1999).

Briefly, the two molecules that serve as transmitters in the adrenergic system are norepinephrine (also called noradrenalin) and epinephrine (also called adrenalin). The receptors that are triggered by these molecules are divided into two classes, designated as alpha (α) and beta (β).

β-adrenergic receptors are not of any particular interest herein; they are reviewed in, e.g., Strosberg 1995 and Liggett 2000.

α-adrenergic receptors are subdivided into the α1 and α2 classes. The α2 subclass, which is of interest herein, has been further subdivided into A, B, C, and D classes, but “little is currently known about the specific function mediated by the various subtypes” (Bylund 1995). Reviews that focus on the α2 receptor system include Stometta et al 1995, Saunders et a 1999, and Rosin 2000. In addition, reviews that focus on drugs which react with α1 and/or α2 receptor subtypes include Eisenach et al 1996, Kamibayashi et al 1997 and 2000, Newcorn et al 1998, Frishman et al 1999, and Cotecchia et al 2000.

Two facts about α2 adrenergic agonist drugs (referred to herein simply as α2 agonists, for convenience) should be recognized and emphasized, since both facts are directly relevant to this invention. One fact is this: α2 agonist drugs have sedating effects; indeed, they are often referred to as “sedative-hypnotic” drugs (e.g., Levanen et al 1996). As such, they are used in both veterinary and human surgery, to help boost the depth and quality of anesthesia provided by a drug such as ketamine (e.g., Verstegen et al 1989; Nevalainen et al 1989; Moens et al 1990; Levanen et al 1996; Handa et al 2000). The ability to relax a patient or pet, and take them deeper into anesthesia while also minimizing certain unpleasant side effects of ketamine anesthesia (such as psychotic “emergence reactions”) is useful and desirable, in anesthesia. However, it becomes a serious problem if a “sedating-hypnotic” drug must be taken every day to control chronic pain. The last thing a patient wants, when struggling with chronic pain, is to feel groggy, sedated, and semi-stuporous every day.

The second fact is this: α2 agonists substantially lower blood pressure. That is their main therapeutic use, i.e., reducing blood pressure in patients suffering from hypertension (high blood pressure). Reduced blood pressure can be useful in someone who needs it, but it can be very dangerous, and potentially even lethal, in someone who doesn't need it. Among other things, reduced blood pressure causes dizziness, which can be dangerous for anyone, and which is especially dangerous in elderly patients, since dizziness can lead to a fall, and a fall by an elderly patient often results in a broken hip or other severe injury.

Accordingly, both of those two facts teach directly away from the current invention, which involves the chronic use of α2 agonists to help control neuropathic pain.

When the Applicants began their research, there was no reason to think that α2 adrenergic agonists could offer a non-sedating drug that could be taken daily, since they are fairly potent sedative-hypnotic drugs. The fact that α2 agonist drugs are sedative-hypnotic agents, potent enough to help carry patients through surgery, teaches strongly and directly away from their daily use, for treating chronic pain without causing unwanted sedation.

Accordingly, one object of this invention is to disclose that when an α2 adrenergic agonist (such as clonidine) is coadministered along with memantine (or possibly other adamantane derivatives, with similar structures and properties), these two agents mutually potentiate one another's ability to relieve neuropathic pain, in a manner which provides sustained and effective neuropathic pain relief, at a low dose of each agent that is below its threshold for producing sedation or other unwanted side effects.

Nnother object of this invention is to disclose a unitary dosage form (such as a tablet, capsule, or skin patch) containing both memantine (or possibly other adamantane derivatives with similar properties) and an α2 adrenergic agonist drug, where each drug is present at a dosage which allows the combination to effectively control neuropathic or other chronic pain, without causing sedation, excessive reduction of blood pressure, cognitive or memory disruptions, or other undesired neurological or physiological side effects.

These and other objects of this invention will become more apparent through the following summary, drawings, and description of the preferred embodiments.

### SUMMARY OF THE INVENTION

This invention discloses that a combination of two drugs, from two different and previously unrelated categories, provides effective and long-lasting relief from neuropathic pain. Both drugs can be taken orally, in a convenient, painless, non-invasive manner that does not require injections.

One drug in this combination is an α2 adrenergic agonist, exemplified by clonidine. These drugs, normally used to control high blood pressure, have been used in a few cases for pain relief, but only by means of intrathecal injection (i.e., injection directly into spinal cord fluid, to avoid dangerous and potentially lethal reductions in blood pressure, which might occur if a high dose of an α2 agonist is injected into non-CNS tissue). They are also known as sedative-hypnotic drugs, and have been used in surgical anesthesia; these traits teaches away from their use on a daily basis, as a non-sedating treatment for chronic pain.

The other drug in the pain-relieving combination is an adamantane derivative which has NMDA antagonist activity, such as memantine.

Tests described herein demonstrate that when memantine is administered together with an α2 adrenergic agonist such as clonidine, these drugs mutually potentiate one another's neuropathic pain-relieving action, and provide potent and sustained neuropathic pain relief, even when each agent is administered at a low dosage that is below its threshold for causing adverse side effects.

Accordingly, these results indicate that combining these two classes of drugs can provide safe and effective relief of neuropathic pain and possibly other types of chronic and/or intractable pain, without serious adverse side effects.

### BRIEF DESCRIPTION OF THE DRAWINGS

FIGS. 1 through 4 are graphs showing the results of tests using a standard model of neuropathic pain, involving the hind legs of rats, and their response to a warming light that shone on the sole of the animal's hindpaw. In all graphs, open circles indicate data from “ligated” limbs, in which the sciatic nerve was chronically irritated by a ligature (a loop of suture material) that was surgically emplaced around the nerve and then tightened. Filled circles indicate “control” data, from sham-operated limbs that did not have any ligatures to cause chronic irritation. Rapid lifting of a ligated paw indicates that the paw was hyper-sensitive.

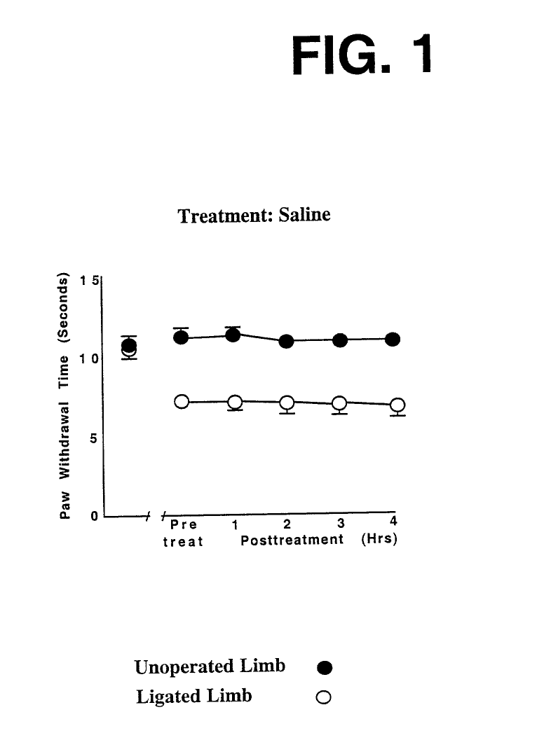

In graphs where the open circles are substantially lower than the filled circles, the drug treatment was not adequate and effective in controlling and reducing the neuropathic pain response. An example is provided in FIG. 1, which is a “baseline” control using a saline injection rather than a drug.

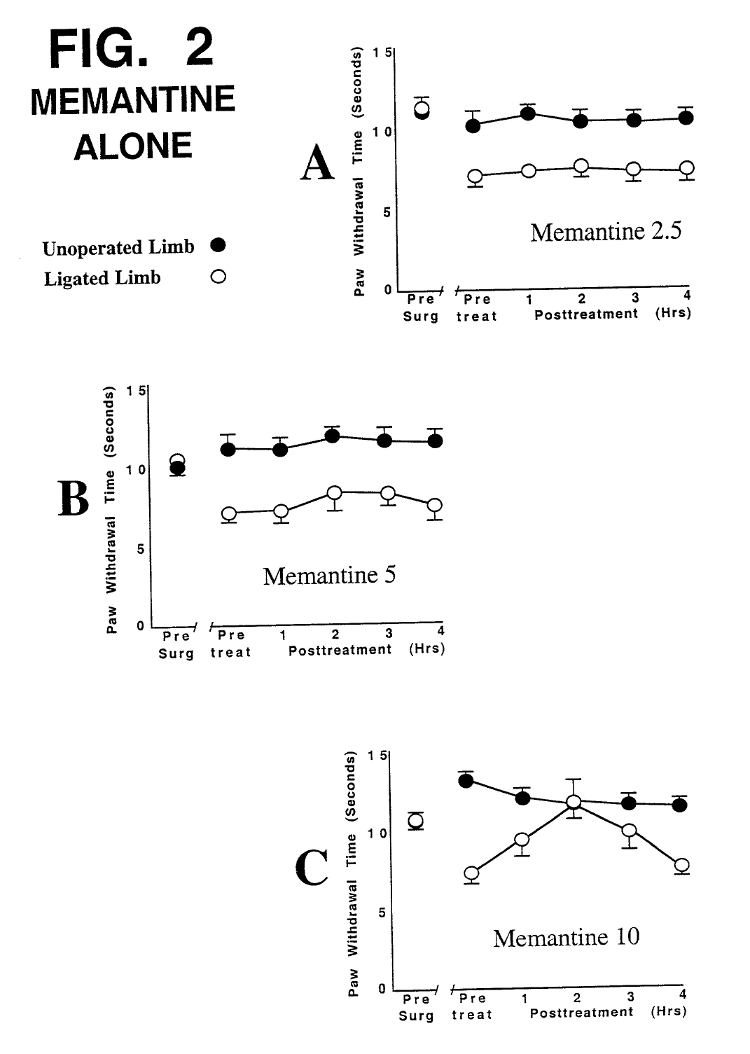

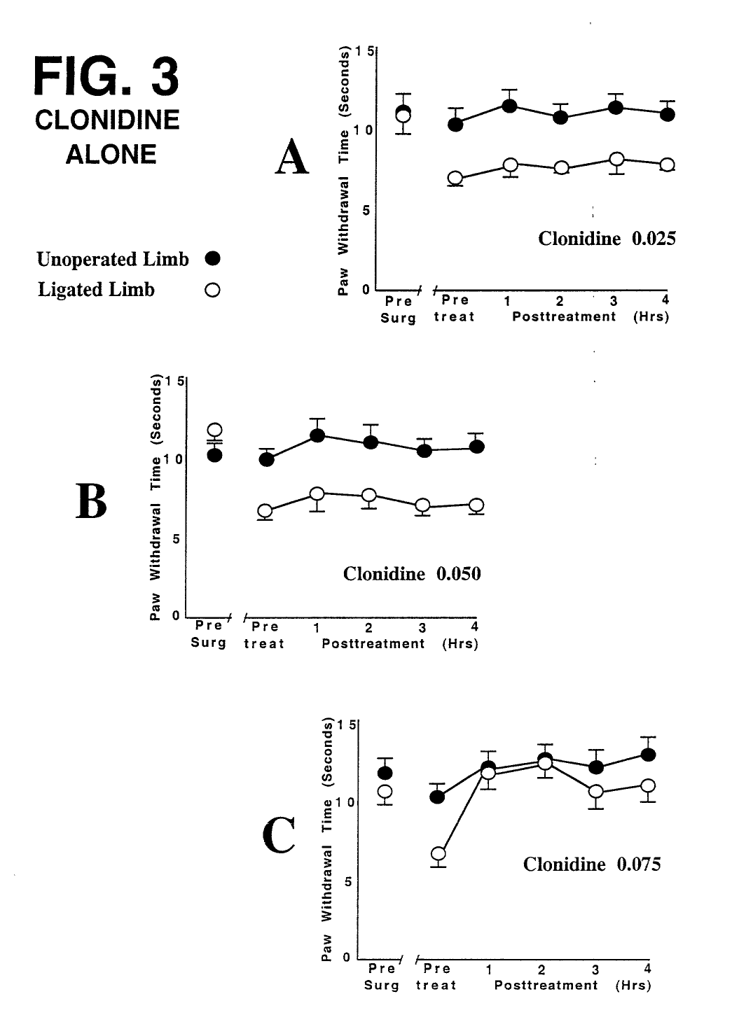

If a drug treatment was effective in reducing and controlling the neuropathic pain response, the vertical gap between a dark circle (non-ligated limb) and an open circle (ligated limb) was relatively small. The graphs in FIGS. 2 and 3 show that procyclidine and clonidine each provided some level of pain relief, but only at high dosages which caused behavioral side effects, and only for a short time.

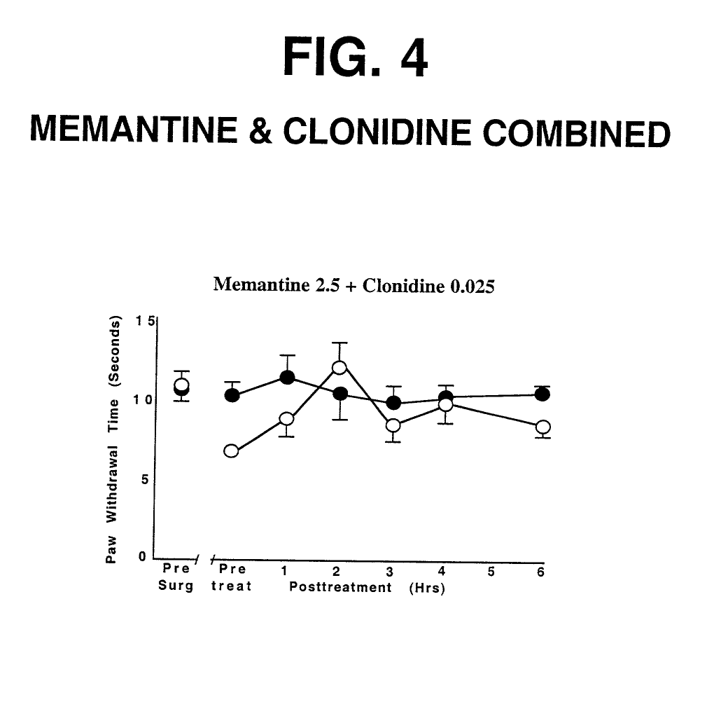

The graphs in FIG. 4 show that combinations of procyclidine and clonidine provided much better and longer-lasting pain relief than either drug could provide by itself. In addition, comparison of the effective dosages in these tests against the same dosages shown in FIGS. 2 and 3 show that the combination worked well even at low dosages of both agents.

### DESCRIPTION OF THE PREFERRED EMBODIMENTS

This invention involves the use of a drug combination for treating chronic and/or intractable pain, such as neuropathic pain. The drug combination requires two distinct types of drugs, which are: (1) memantine, or a similar adamantane analog or derivative which has NMDA antagonist activity; and, (2) an α2 adrenergic agonist drug, such as clonidine, guanabenz, and lofexidine.

These drugs are likely to be used over long spans of time, such as months or even years, possibly for the entire remaining life of the patient. Instead of being a “cure” for neuropathic or other chronic pain, which would permanently alter and correct damaged or dysfunctional neuronal circuitry that causes the neuropathic or other chronic pain, this invention discloses a treatment and control regimen, which can help the affected patient suppress and control the chronic pain, with minimal unwanted side effects such as sedation.

Since these drugs are likely to be used on a daily basis (ranging from once per day, up to several times per day, depending on the severity of the condition being treated), both drugs should be in a form suited for noninvasive and painless adminstration. Such modes of administration include, for example, oral ingestion of a tablet, capsule, or liquid, or use of a skin patch or other “transmembrane” route.

In addition to clonidine (a well-known α2 agonist, used to generate the test results shown in the Figures), various other known drugs have been developed which have selective affinity for α2 adrenergic receptors. Such drugs include iodoclonidine, guanabenz, guanfacine, xylazine, lofexidine, medetomidine, dexmedetomidine, tizanidine, rilmenidine, azepexole, α-methyldopa, and α-methyl-noradrenaline. Any of these drugs, or any other α2 adrenergic agonist which is currently known or discovered in the future, can be tested for efficacy in reducing and controlling neuropathic pain or any other form of chronic and/or intractable pain, when coadministered with an adamantane derivative having NMDA antagonist activity.

In general, “selective” or “highly selective” α2 agonists are preferred, since they pose lower dangers of unwanted side effects. For example, clonidine is regarded as a selective α2 agonist, since its affinity for α2 adrenergic receptors is significantly higher than its affinity for α1 adrenergic receptors. However, clonidine is also known to have significant affinity for α1 receptors.

Accordingly, clonidine (which is one of the most well-known and widely used α2 agonist drugs) is regarded herein as a “benchmark” drug. It is a “selective” α2 agonist drug, but it is not regarded as a “highly selective” α2 agonist drug. The phrase “highly selective α2 agonist” refers to an α2 adrenergic agonist drug which has substantially higher selectivity for α2 receptors (and substantially lower affinity for α1 receptors) than clonidine. Published reports have stated that such “highly selective” α2 agonists include dexmedetomidine, guanfacine, and azepexole, and may also include lofexidine and various other α2 agonists as well.

In addition, it must be recognized that a major physiological effect of clonidine (and most other known α2 agonist drugs) is their ability to reduce blood pressure. That is their normal medical use, and it is quite useful in patients suffering from hypertension (high blood pressure). However, reductions in blood pressure are likely to be unwanted, and even potentially dangerous, in patients who do not suffer from hypertension, and who instead are suffering from neuropathic or other chronic pain. Accordingly, the phrase “α2 agonist which has little hypotensive side effect” refers to an α2 agonist which has less activity than clonidine, in reducing blood pressure. Published reports indicate that lofexidine and guanfacine both fall within this criterion, and various other α2 agonists may also qualify.

Although the Applicants are not aware of any publication that systematically ranks or quantifies numerous α2 agonists in terms of α2 selectivity, potency in reducing blood pressure, severity or absence of side effects, and ability to penetrate mammalian blood-brain barriers, such information is provided, in scattered form, in various locations (e.g., Goodman and Gilman 1990 at page 308; Ruffolo et al 1993 at page 264; Doze et al 1989 at page 75). In addition, evaluation of these relevant properties in any known or hereafter-discovered α2 agonist can be carried out with routine experimentation, using conventional procedures. Such tests include competitive binding assays to evaluate selectivity for α1 and α2 receptors; tests on both normal and hypertensive lab animals, to evaluate hypotensive activity; and analysis of CNS tissue following administration of radiolabelled drugs, to evaluate BBB permeability. Accordingly, based on the teachings herein, methods are provided for screening the α2 agonist drugs listed herein, or any other α2 agonist drugs which are currently known or hereafter discovered, and determining which ones will have the best profile of benefits vs. unwanted side effects, when coadministered with an adamantane abalog or derivative which has NMDA antagonist activity, to relieve neuropathic or other chronic pain as disclosed herein.

It is not asserted that the drug combinations disclosed herein will be completely free of any and all side effects, in all patients. Instead, this disclosure asserts that: (i) this drug combination allows the use of relatively low dosages of both constituent drugs, when they are coadministered with each other; and, (ii) the use of relatively low dosages of both constituent drugs strongly indicates that any adverse side effects will be minimized, and will be fully acceptable to large numbers of patients. Indeed, one of the most remarkable and valuable traits of the drug combination disclosed herein is that these two classes of drugs, when administered together, appear to be highly potent and effective in reducing neuropathic pain, even when each drug is administered at a dosage which is only a fraction of the dosage that begins to exert detectable behavioral or other effects in treated animals.

As used herein, the phrase “unacceptable side effects” refers to side effects which, in a particular patient or class of patients, rise to a level of unpleasantness which that patient (or class of patients) regards as outweighing the benefits of the treatment. Clearly, dosages and frequency of administration will vary substantially between different patients; patients who are suffering from nearly unbearable intractable pain are likely to take high doses of both drugs, four or more times each day; by contrast, patients who are suffering from minor annoyances will take lower dosages, only once or twice each day. Accordingly, the acceptability of any side effects a drug combination as disclosed herein might cause, in a particular patient or class of patients, must be viewed in light of the pain-relieving benefits it provides for that patient or class of patients.

Animal Test Design and Results

All of the graphs in FIGS. 1-4 pertain to the effects of various drug treatments on measurable pain-sensitive responses in test animals (rats). FIG. 1 shows the results of saline controls, which provided no pain relief. FIGS. 2 and 3 displays the results of a single drug (memantine or clonidine). FIG. 4 displays the results of the memantine-plus-clonidine combination, in two different dosages.

In all experiments, one hind limb of the rat has undergone a surgical operation in which a loop (“ligature”) of suture material was placed around the sciatic nerve and gently tightened. Over the course of several days, this caused chronic irritation to the nerve, driving it into a “hyper-sensitized” condition of the type which occurs in damaged neuronal circuits that cause neuropathic pain in humans. The other hind limb of each rat did not receive any operation, and served as a control condition.

After the nerve ligation operation, a waiting period of 8 days elapsed, to allow the ligated limb to reach a peak of hypersensitized chronic irritation of the ligated sciatic nerve. This sciatic nerve model was first described by Bennet and Xie 1988, and is widely regarded as a useful animal model for studying neuropathic pain.

To test the degree of sensitivity in the ligated limb vs. the unoperated limb, a beam of light (thermal stimulus) is applied to the soles (i.e., the undersurface area which is not covered by fur) of the hind paws, by shining a focused beam of light onto the sole, through the glass floor of a testing chamber. This beam was sufficiently hot to generate some discomfort, without posing any risk of burning the paw. The number of seconds that elapsed after commencement of the thermal stimulus, until the rat lifted up the paw, were detected electronically and recorded automatically.

Each limb is tested initially for thermal pain sensitivity, before the ligation operation is performed (pre-surgery), to ensure that both limbs had the same degree of pain sensitivity. After 8 days following surgery, each limb was tested again, before any treatment was given (pre-treatment), to ensure that the nerve ligature successfully provoked a hyper-irritable condition in the ligated limb. After drug treatment (at time=0, on the graphs), each limb was tested for thermal pain sensitivity at hourly intervals, for 4 hours.

The graph in FIG. 1 depicts the level of pain sensitivity in each limb under control conditions, when a saline injection instead of an active drug was administered. In all graphs, open circles indicate data from ligated limbs, and filled circles indicate control data, from sham-operated limbs that did not have any ligatures to cause chronic irritation.

Rapid lifting of a ligated paw indicates that the paw was hyper-sensitive. If a large vertical gap occurs between the ligated limb (open circle) and the control limb (closed circle), such as shown in FIG. 1 at all times, this indicates that (i) the ligated paw was indeed suffering from a hyper-irritable neuropathic pain condition, and (ii) the drug treatment was not effective in reducing and controlling the response to the painful stimulus. After saline (control) treatment, there was no change in pain sensitivity in either limb, as indicated by the fact that the open circles (ligated limbs) and dark circles (unoperated limbs) continued to be widely separated, consistently showing more rapid paw withdrawals in the ligated limbs, following thermal stimulus, at each hourly interval.

The graphs in FIG. 2 show that intraperitoneal injection of memantine, at three different dosages, did not provide adequate and lasting relief from pain sensitivity. At the lowest dosage used (2.5 mg/kg, shown in FIG. 2A), the memantine had no apparent effect at all. At 5 mg/kg, shown in FIG. 2B, the memantine had some effect at 2 and 3 hours post-treatment, but that response in ligated limbs did not reach or even closely approach the response times in unoperated limbs; in addition, even that mild effect shown had dropped off substantially by 4 hours post-treatment.

At the highest dosage tested (10 mg/kg, shown in FIG. 2C), relief from neuropathic pain built up to a peak of maximum and effective oain relief, at the 2 hour testing time. However, this level of pain relief dropped off substantially by the third hour, and returned to the untreated baseline level by 4 hours post-treatment.

The graphs in FIGS. 3A, 3B and 3C show the pain sensitivity in each limb, following intraperitoneal injection of clonidine at 0.025 mg/kg, 0.05 mg/kg or 0.075 mg/kg, respectively. At the two lower doses, clonidine showed no significant relief of pain. At the highest dose, there was significant and relatively sustained neuropathic pain relief; however, at that dosage of clonidine, the rats displayed significant sedation and loss of motor activity and control, as described in Example 5. In at least some and probably even most humans, a comparable dose of clonidine would very likely produce serious unwanted sedation, and a lowering of blood pressure to an unacceptable and potentially dangerous degree.

The graph in FIG. 4 shows that when memantine and clonidine were administered together, even at low dosages, the combination provided sustained relief of neuropathic pain, out to a 6-hour test time, when the tests were terminated. Just as importantly, these graphs show that effective and sustained pain relief was provided at low dosages of only 2.5 mg/kg memantine and 0.025 mg/kg clonidine; neither of those dosages caused any significant reduction in pain when that drug was administered by itself at that dosage.

Modes of Administration

The compositions of this invention comprise mixtures of an α2 adrenergic agonist, with an adamantane drug (such as memantine) which has antagonist activity at NMDA receptors. The two components act together in a synergistic and potentiating manner, causing the mixture to provide better and longer lasting non-sedating pain relief than either drug can provide without the other drug. This allows each component to be administered at a relatively low dosage, and avoids or minimizes any undesired side effects.

Both compounds must be “pharmaceutically acceptable”; this embraces the traits which make a drug suitable and practical for administration to humans, or to non-human animals if used in veterinary medicine. For example, such compounds must be sufficiently chemically stable to have an adequate shelf life under reasonable storage conditions, and they must be physiologically acceptable when introduced into the body by a suitable route of administration. The term “pharmaceutically acceptable” does not require a complete absence of any toxicity or unwanted side effects; instead, it implies that any toxic or other unwanted effects must be tolerable, and must be outweighed by some benefit which the drug can provide for at least some patients or animals.

The compositions of this invention may be administered by any suitable route which will introduce the intended drug(s) into the bloodstream. As noted above, preferred modes should use painless non-invasive means, such as oral ingestion of tablets, capsules, or liquids, or transmembrane routes, such as skin patches, nasal sprays, lozenges, penetrating ointments, etc. Intramuscular, intravenous, or other forms of injection, as well as subcutaneous implantation of slow-release devices or formulations or osmotic mini-pumps, are also possible, but they are less convenient and more painful and troublesome than noninvasive modes such as pills or skin patches.

All such modes of administration are all well known in the pharmaceutical arts, and typically require a pharmaceutically acceptable carrier in addition to the active ingredients. In making the pharmaceutical mixtures disclosed and claimed herein, the active ingredients will usually be mixed with and diluted by a carrier, or enclosed within a carrier such as a capsule. When the carrier serves as a diluent, it may be a solid, semisolid or liquid material which acts as a vehicle, excipient or medium for the active ingredients. Thus the composition can be in the form of tablets, pills, powders, lozenges, chewing gum, cachets, elixirs, emulsions, solutions, syrups, suspensions, aerosols (as a solid or in a liquid medium), ointments containing for example up to ten percent by weight of the active compound, soft and hard gelatin capsules, suppositories, sterile injectable solutions, and sterile packaged powders.

For oral administration, the compositions of this invention can be admixed with carriers and diluents molded or pressed into tablets, or enclosed in gelatin capsules, or otherwise loaded into digestible plastic or other capsules. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propyl-hydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient, by employing procedures well known in the art.

The compositions are preferably formulated or packaged in a unit dosage form, each dosage unit containing an effective amount of both (i) one or more alpha-2 adrenergic receptor agonists, and (ii) memantine or another adamantane compound with NMDA antagonist activity. The term “unitary dosage form”, as used herein, has two meanings. One meaning refers to physically discrete units (such as capsules, tablets, or skin patches) which are suitable as unitary dosages for human subjects (or for pets, etc.) wherein each unit (each pill, skin patch, etc.) contains a predetermined quantity of each of the two active drugs discussed herein. In this type of unitary dosage form, the dosages of both drugs are predetermined, and are designed to safely produce the desired therapeutic effects, in most patients.

The second meaning of “unitary dosage form” refers to a liquid, dry powder, or similar formulation which is not divided into discrete units, but which contains each of the two active drugs discussed herein at predetermined concentrations which allow desired dosages of both drugs to be dispensed conveniently by referring to a fixed volume of the liquid or powder. As one example, a liquid containing two active drugs in a carrier can be provided in a unitary dosage form, if the desired dosage of each drug can be taken by ingesting a fixed quantity (such as a single teaspoon, tablespoon, etc.) of the liquid. Similarly, a powdered formulation of two drugs can be provided in a unitary dosage form, if the desired dosage of each drug can be taken by stirring a teaspoon, tablespoon, or “scoop” (if a plastic scoop is provided in the container) of the material into a suitable liquid which is then swallowed.

Alternately or additionally, non-divided preparations such as liquids, aerosols, or powders can be packaged inside unitary-dosage container devices which are designed to dispense a predetermined volume of material each time the device is used. Examples of unitary-dosage dispensing containers include: (i) inhaler devices, which release a predetermined amount of medicine (such as for treating asthma) into the mouth or nasal sinuses each time the device is squeezed; and, (ii) plastic bottles which are provided with a small chamber near the outlet, wherein the chamber is provided with a liquid-level marker that indicates how much liquid should be squeezed into the chamber for a proper dose of the liquid. By controlling the concentration of each drug in the liquid, aerosol, powder, or other preparation in a dispenser, unitary dosage of drug mixtures can be provided by such devices.

In either case, a “unitary dosage form” of a non-divided material such as a liquid, aerosol, or powder can be provided by controlling the concentrations of both drugs carried by the material, so that administration of a controlled volume of the material will provide the desired unitary dosage.

The amount of either drug, in a unitary dosage formulation, will depend on factors such as the severity of the pain condition being treated in a particular patient, and the the amount and potency of the other drug in the same formulation. These drug cocmbinations are (or are likely to be) available only with a prescription from a physician; they are not available over-the-counter. Accordingly, the preferred dosage and mode of administration of the two drugs, in combination, will be under the control of a qualified physician, who can evaluate all relevant factors for any specific patient (including the age, weight, and response of the individual patient, the severity of the patient's symptoms, the chosen route of administration, etc.). In addition, it is standard practice in treatments of this nature for a physician to administer an initial dosage for a test period such as two weeks, and subsequently adjust the dosage depending on the results observed during the test period. Therefore, the dosage ranges provided below are intended to provide general guidance and support for the teachings herein, but are not intended to limit the scope of the invention.

In general, the preferred dosage of an α2 agonist will usually lie within the range of from about 0.001 to about 1000 mg, more usually from about 0.01 to about 500 mg per day. The amount of the adamantane drug will typically be within the range of from about 0.001 to about 1000 mg, more usually from about 0.01 to about 500 mg per day. Alternately, expressed in terms of mg/kg (i.e., milligrams of drug per kilogram of patient body weight), the dosage of α2 agonist normally will fall within a range of about 0.00002 to about 50 mg/kg of body weight, and most commonly will fall within a range of about 0.0002 to about 20 mg/kg body weight, while the dosage of the adamantane drug will normally fall within a range of from about 0.00002 to about 50 mg/kg of body weight, and most commonly within a range of about 0.0002 to about 20 mg/kg body weight.

Pro-Drugs, Salts, Isomers and Analogs

The terms, “drug”, “agonist”, and “antagonist,” as used herein, includes so-called “pro-drugs” which are administered in a form that is known and intended to be metabolized, inside a patient's body, into a different form which has a specific desired activity. As an example, α-methyldopa is a pro-drug form of an α2 agonist; the dopa form is actively transported into the CNS, which is desirable, and then enzymes inside the CNS convert the dopa form into α-methyl-norepinephrine, which is the active α2 agonist form of the drug. In such cases, both α-methyldopa and α-methyl-norepinephrine would be regarded as α2 agonist drugs for the purposes of this invention.

Various salts and isomers (including stereoisomers) of the drugs listed herein can be used. The term “salts” can include alkali metal salts as well as addition salts of free acids or free bases. Examples of acids which may be employed to form pharmaceutically acceptable acid addition salts include inorganic acids such as hydrochloric, sulfuric, or phosphoric acid, and organic acids such as acetic, maleic, succinic, or citric acid, etc. Alkali metal salts or alkaline earth metal salts include, for example, sodium, potassium, calcium, or magnesium salts. All of these salts (or other similar salts) may be prepared by conventional means. The nature of the salt or isomer is not critical, provided that it is non-toxic and does not substantially interfere with the desired pharmacological activity.

The term “analog” is used herein in the conventional pharmaceutical sense, to refer to a molecule that structurally resembles a referent molecule (such as adamantane or memantine), but which has been modified in a targeted and controlled manner to replace one or more specific substituents of the referent molecule with an alternate substituent, thereby generating a molecule which is structurally similar to the referent molecule. Synthesis and screening of analogs, to identify slightly modified versions of a known compound which may have slightly improved traits (such as higher potency and/or selectivity at a specific targeted receptor type, greater ability to penetrate mammalian blood-brain barriers, etc.) is an approach that is well known in pharmaceutical chemistry.

Similarly, the term “adamantane derivative” is used herein to describe a compound which is derived from adamantane, i.e., adamantane (or an available derivative thereof, such as mementine) is used as a starting reagent or intermediate, in the process used to create a similar but slightly different drug which resembles adamantane or memantine.

Adamantane analogs and derivatives are covered by the claims below, but only if and to the extent that they function as pharmaceutically acceptable NMDA antagonists, and show good therapeutic efficacy against neuropathic pain when co-administered with an α2 adrenergic agonist drug.

In addition, analogs and derivatives of α2 agonist drugs, and of adamantane drugs which suppress activity at NMDA receptors, can be created which have either greater or lower abilities to permeate blood-brain barriers, using methods known to those skilled in the art. Such drugs can be tested, using the methods disclosed herein, to determine whether such variants having either higher or lower BBB permeation rates have improved therapeutic efficacy (which may include greater potency or duration, fewer side effects, etc.) in controlling neuropathic pain, when used as disclosed herein.

### EXAMPLES

### Example 1

### Test Procedures and Control Results

The first set of in vivo tests involved sciatic nerve ligation in rats, using procedures described in Bennett and Xie, Pain 33: 87-107 (1988). This test involves surgically placing and tightening a loop of suture material around the sciatic nerve in one hind leg of a rat; the other hind leg serves as a control. Within about a week, the ligated sciatic nerve becomes irritated and reaches a hyper-responsive “kindled” state, where it will respond rapidly to even a mild stimulus that is not painful to an untampered leg. As such, it offers a model of what happens in pain pathways that have become pathologically hypersensitized in a human suffering from neuropathic pain.

In these tests, on the 8th day after surgery, the rat is placed in a testing device which electronically measures how quickly or slowly it acts, in lifting up a paw in response to a standardized mild warming (thermal) stimulus. The warming stimulus is generated by a light beam which shines onto the bottom surface of the paw, through the glass floor of the testing enclosure. The device is electronically controlled and automated, and measurements are made of the number of seconds that elapse after the light beam begins to shine on the bottom of the paw, until the rat lifts its paw.

To ensure that the surgical operation itself did not affect the outcomes, comparative tests using each dosage level were also carried out on control populations of “sham-operated” animals. These animals were subjected to the same type of anesthesia and incisions on one limb, but no loop of suture material was placed around the sciatic nerve.

In all animals, the other hind limb was not operated on, and provided an additional control, generated by the same animal.

The results of these control experiments (not shown) demonstrated that sham-operated limbs in control animals, and unoperated limbs in test animals, responded identically to the thermal stimulus, under all treatment conditions.

To establish baseline values for pain sensitivity of each limb in each group of animals, each limb was tested initially for thermal pain sensitivity before the ligation operation was performed (pre-surgery). After 8 days, each limb was again tested before any drug treatment was administered (shown by the “pre-treatment” legend in all graphs). After drug administration, each limb was tested for thermal pain sensitivity at hourly intervals, for 4 hours.

Saline controls and all drugs were administered by intraperitoneal (IP) injection, i.e., hypodermic injection into the abdominal region. This is a convenient and reliable method of drug administration, for tests involving rats.

The test results are shown graphically in the Figures; each circle indicates the mean value, and the vertical bars show the standard error of the mean (SEM). In these graphs, the vertical position of each open circle indicates how many seconds elapsed (on average) between the commencement of the light beam which warmed the lower surface of the foot, and the act of lifting the hyper-sensitized hindpaw having the ligated nerve; by comparison, the vertical position of each darkened circle indicates how many seconds elapsed (on average) between the beginning of the thermal stimulus and lifting of the hind paw of the unoperated limb. Accordingly, a large vertical separation between a light circle (nerve-ligated paw) and a dark circle (control paw) indicates that the test animals were not well protected against neuropathic pain by the drug(s) used, at that dosage. By contrast, closely aligned dark and light circles indicate that the test animals were effectively protected against neuropathic pain by the drug(s) at the indicated dosage(s), at that point in time.

Large vertical gaps, showing a lack of effective pain control, are clearly indicated by the use of saline controls to test nerve-ligated animals, as shown in FIG. 1. Failure of the open circles to move vertically upward at any post-treatment interval, in a manner which reduced or closed the gap between the open circles and the dark circles, indicates that the inactive saline treatment did not provide any protection against neuropathic pain, in the nerve-ligated limb.

### Example 2

### Memantine Alone

The graphs in FIG. 2 show that IP injection of memantine alone, at 2.5 mg/kg (FIG. 2A) did not significantly change the pain sensitivity in either limb, as evidenced by the fact that both the open circles and dark circles remain at their pre-treatment vertical levels at each post-treatment interval. At all times, the gap between the open and dark circles remained widely separated.

At twice that dosage, 5 mg/kg (FIG. 2B), memantine showed some slight effects in reducing neuropathic pain, at the 2nd and 3 hours post-treatment. That effect had worn off completely by the 4th hour.

The graph in FIG. 2C shows that IP injection of memantine at 10 mg/kg caused a climb to a peak of highly effective pain relief at 2 hours post-treatment; however, that pain relief had dropped off substantially by the 3rd hour, and had disappeared completely by the 4th hour.

No higher doses were tested, since it was felt by the Applicants that even the 10 mg/kg dosage (which was nearly half the dosage that began to show serious neurotoxic side effects in the brains of other treated animals) was already too high to offer a feasible and practical daily treatment for chronic pain.

### Example 3

### Clonidine Alone

Clonidine also was tested at several dosages, by itself, using the same procedures described in Example 1. The graphs in FIGS. 3A, 3B and 3C show the pain sensitivity in each limb when the treatment used was IP injection of clonidine at 0.025 mg/kg, 0.05 mg/kg or 0.075 mg/kg, respectively.

At the two lower doses (0.025 mg/kg and 0.05 mg/kg), clonidine showed no significant relief of neuropathic pain.

At the highest dose tested (0.075 mg/kg), significant and relatively sustained neuropathic pain relief was shown. However, at that dosage, the rats displayed significant sedation. In humans, a comparable dose would very likely produce both severe sedation, and a lowering of blood pressure to an unacceptable and potentially dangerous degree.

### Example 4

### Combination of Memantine and Clonidine

Using the same testing procedures described above, the effects of co-administering clonidine at a dose of 0.025 mg/kg, together with memantine at a dose of 2.5 mg/kg were evaluated.

The results, shown in FIG. 4, clearly indicate that when memantine and clonidine were administered together, the combination was highly effective in providing sustained relief from neuropathic pain. Indeed, the extent of pain relief was still so high after 4 hours that additional testing was repeated after 6 hours, at which time the pain reduction was still significantly better than the pre-treatment baseline.

In this test, each drug was administered at a dose that, by itself, did not offer any detectable pain relieving effects, and did not pose any serious risk of neurotoxic side effects, or cognitive or behavioral impairments. The 2.5 mg/kg dosage of memantine corresponds to FIG. 2A, which clearly shows that at that 2.5 mg/kg, memantine by itself offered no protection whatever against neuropathic pain. The 0.025 mg/kg dosage of clonidine corresponds to FIG. 3A, was shows that, at that dosage, clonidine by itself also was totally ineffective in providing any relief from neuropathic pain.

Example 5

### Reflex Testing to Confirm Hindpaw Control

In the experiments described in Examples 1-4, above, eight days after the rats had undergone surgical placement of a ligature around the sciatic nerve, and before they were subjected to drug treatment or a thermal pain stimulus, each animal was tested for the presence of certain types of reflexes that must be intact in order for the rat to promptly lift its paw in response to a stimulus.

One such evaluation is performed by holding the animal by its torso, with its lower limbs hanging downward, and bringing the top surface of a hind paw in contact with the edge of a table top. All animals with an intact placing reflex will immediately lift its hindpaw and place it on top of the table, in response to this stimulus. Performance of this test ensures that the ligature around the sciatic nerve has not impaired the animal's ability to lift its paw. If any animal was unable to perform that function without impairment, it was removed from further testing.

### Example 6

### Additional Tests to Evaluate Adverse Side Effects

Studies were performed on separate groups of rats (not used in the pain testing sessions), to determine whether drug treatments at the doses administered in Example 2-4 would produce detectable impairments to reflex response, motor control, or behavior. These studies helped clarify whether the drug dosages that proved successful in relieving neuropathic pain might produce adverse side effects that would preclude using this drug combination in humans for the relief of chronic pain.

To address these issues, rats with sciatic nerve ligations, and rats with only sham sciatic nerve ligations, were treated with various doses of memantine, clonidine, or the two agents together. They were then evaluated, using the following battery of sensorimotor and activity tests:

Sensorimotor Battery: Testing was done at 1 hour post-treatment, which corresponded with the time of peak effect of drugs on paw withdrawal latency. Briefly, the sensorimotor battery consisted of five tests (Wozniak et al 1990) that are known to be able to detect significant muscle weakness, incoordination, somnolence, or other behavioral impairments.

For all of the tests in this sensorimotor battery, all treatment groups first received a habituation trial, to allow each rat to acclimate to the test situation. In all tests, protective padding was placed under any elevated apparatuses, to prevent pain or injury from falling. The tests performed were as follows:

(1) Plank test. A rat was timed for how long it could remain on a wooden plank that is 3 cm wide, elevated 61 cm above the floor.

(2) Walking initiation (also a test of sedation and activity level). Each rat was placed in the middle of a square outlined by white cloth tape (50×50 cm) on a smooth black surface of a large table top, and was timed for how long was required for the rat to leave the square (place all four paws outside the square).

(3) Platform. Each rat was timed for how long it could remain perched on an elevated (61 cm above the floor) platform of small dimensions (7.6×15.2 cm).

(4) Inclined screen. Each rat was placed in the middle of an elevated (61 cm above the floor) wire mesh grid (8 squares per 10 cm) which was inclined at 60 degrees with the floor. The rat's head was oriented downward toward the floor, and it was timed for how long it could continue to hold onto the screen.

(5) One hour activity level. Locomotor activity was evaluated beginning at 0.5 hr following drug treatment, and lasting for 1 hr. In this test, the rat was placed in a cage equipped with three pairs of photoelectric cells, placed at regular intervals across the width of the cage, so that the animal's activity was monitored by automatically recording the number of “beam breaks” registered as the animal moved about the cage.

The results indicated that clonidine, administered by itself at a dose of 0.050 mg/kg, did reduce activity levels, but only slightly and to a barely significant degree. At a dose of 0.075 mg/kg, clonidine substantially reduced activity levels, indicating that at that does, it had substantial sedating effects.

Memantine, at the 2.5 mg/kg dosage used in Examples 2 and 4, and shown in FIGS. 2A and 4, did not cause any detectable impairments on any of the above described sensorimotor tests.

Thus, there has been shown and described a new and useful combined drug treatment for reducing neuropathic and/or other chronic pain. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

### REFERENCES

Bennett, G. J., et al, “A peripheral mononeuropathy in rat produces disorders of pain sensation like those seen in man,” Pain 33: 87-107 (1988)

Bylund, D. B., “Pharmacological characteristics of alpha-2 adrenergic receptor subtypes,” Ann N Y Acad Sci 763: 1-7 (1995)

Choi, D. W., “Excitotoxic cell death,” J Neurobiol 23: 1261-1276 (1992)

Cotecchia, S., “The alpha 1a and alpha 1b-adrenergic receptor subtypes: molecular mechanisms of receptor activation and of drug action,” Pharm Acta Helv 74: 173-9 (2000)

Eisenach, J. C., et al, “Alpha-2 adrenergic agonists for regional anesthesia: A clinical review of clonidine,” Anesthesiology 85: 655-74 (1996)

Frishman, W. H., et al, “Alpha-adrenergic blocking drugs in clinical medicine,” J Clin Pharmacol 39: 7-16 (1999)

Goodman and Gilman, eds., The Pharmacological Basis of Therapeutics, fifth edition (Macmillan, 1975)

Goodman and Gilman, eds., The Pharmacological Basis of Therapeutics, eighth edition (Pergamon Press, 1990)

Handa, F., et al, “Effects of oral clonidine premedication on side effects of intravenous ketamine anesthesia,” J Clin Anesth 12: 19-24 (2000)

Jevtovic-Todorovic, V., et al, “Clonidine potentiates the neuropathic pain relieving action of MK-801 while preventing its neurotoxic and hyperactivity side effects,” Brain Res 781: 202-211 (1998)

Kamibayashi, T., et al, “Alpha-2 adrenergic agonists,” Can J Anaesth. 44: R13-22 (1997)

Kamibayashi T, et al, “Clinical uses of alpha2-adrenergic agonists,” Anesthesiology 93: 1345-9 (2000)

Levanen, J., “Dexmedetomidine premedication attenuates ketamine-induced cardiostimulatory effects and postanesthetic delirium,” Anesthesiology 82: 1117-25 (1995)

Liggett S. B., “Pharmacogenetics of beta-1-and beta-2-adrenergic receptors,” Pharmacology 61: 167-73 (2000)

Louis, W. J. et al, “Sites of action of alpha-2 agonists in the brain and periphery,” Am. J. Cardiol. 61: 15D-17D (1988)

Moens, Y., et al, “A comparative study of medetomidine/ ketamine and xylazine/ketamine anesthesia in dogs,” The Veterinary Record 127: 567-571 (1990)

Nevalainen, T., et al, “Evaluation of anesthetic potency of medetomidine-ketamine combination in rats, guinea pigs, and rabbits,” Acta Veterinaria Scandinavica 85: 139 (1989)

Newcorn, J. H., et al, “Alpha 2 adrenergic agonists: neurochemistry, efficacy, and clinical guidelines for use in children,” Pediatr Clin North Am 45: 1099-22 (1998)

Olney, J. W., “Brain lesions, obesity, and other disturbances in mice treated with monosodium glutamate,” Science 164: 719-721 (1969)

Olney, J. W., et al, “Anti-Parkinsonian agents are phencyclidine agonists and N-methyl aspartate antagonists,” Eur. J. Pharmacol. 142: 319-320 (1987)

Olney, J. W., “Excitotoxic amino acids and neuropsychiatric disorders,” pp. 47-71 in Annual Review of Pharmacology and Toxicology, Volume 30 (R. George et al, eds., Annu Rev Inc, Palo Alto, Calif. (1990)

Olney, J. W., et al, “NMDA antagonist neurotoxicity: Mechanism and protection,” Science 254: 1515-1518 (1991)

Rosin, D. L., “Distribution of alpha 2A- and alpha 2C-adrenergic receptor immunoreactivity in the central nervous system,” Methods Mol Biol 126: 475-505 (2000)

Ruffolo, R. R., et al, “Pharmacologic and therapeutic applications of α2-adrenoceptor subtypes,” Annu Rev Pharmacol Toxicol 32: 243-279 (1993)

Saunders, C., et al, “Localization and trafficking of alpha2-adrenergic receptor subtypes in cells and tissues,” Pharmacol Ther. 84: 193-20 (1999)

Stometta, R. L., et al, “Alpha-2 adrenergic receptors: Immunohistochemical localization and role . . . ” Ann N Y Acad Sci. 763: 541-51 (1995)

Strosberg, A. D., “Structure, function, and regulation of the three beta-adrenergic receptors,” Obes Res 3 Suppl 4: 501S-505S (1995)

Verstegen, J., et al, “Medetomidine/ketamine anaesthesia in cats,” Acta Veterinaria Scandinavica 85: 117-123 (1989)

Wozniak, D. F., “Behavioral effects of MK-801 in the rat,” Psychopharmacology 101: 47-56 (1990)

1. A method for treating chronic pain, comprising the step of administering, to a mammal suffering from chronic pain, a drug combination comprising:
(a) an adamantane derivative which is pharmaceutically acceptable, and which suppresses activity at NMDA-type glutamate receptors; and,
(b) a second drug which stimulates activity at alpha-2 adrenergic receptors,
wherein the first and second drugs are administered at dosages which, when combined, provide synergistic and therapeutically effective relief from chronic pain, wherein such relief lasts longer than comparable pain relief provided by either drug alone, and wherein the combination causes lower levels of adverse side effects than either drug administered by itself would cause at a dosage which provides comparable short-term relief from chronic pain.

2. The method according to claim 1 wherein the first drug is selected from the group consisting of memantine, and prodrugs, salts, isomers, analogs and derivatives thereof which are pharmaceutically acceptable and which suppresses activity at NMDA-type glutamate receptors.

3. The method according to claim 1 wherein the second drug is selected from the group consisting of clonidine, iodoclonidine, guanabenz, guanfacine, xylazine, lofexidine, medetomidine, dexmedetomidine, tizanidine, rilmenidine, azepexole, α-methyldopa, and α-methylnoradrenaline, and prodrugs, salts, isomers, analogs and derivatives thereof which are pharmacologically acceptable, and which stimulate activity at α2 adrenergic receptors.

4. The method according to claim 1 wherein each drug is administered on a daily basis, in a painless manner that does not require a hypodermic injection.

5. The method according to claim 4 wherein each drug is administered by means selected from the group consisting of oral ingestion and transmembrane permeation.

6. A pharmacological mixture suited for daily treatment of chronic pain, comprising a combination of:
(a) a first drug which is an adamantane derivative which is pharmaceutically acceptable and which suppresses activity at NMDA-type glutamate receptors; and,
(b) a second drug which stimulates activity at α2 adrenergic receptors,
wherein the first and second drugs are present in the mixture at relative concentrations which, when combined, can be administered to a mammal suffering from chronic pain, in a manner which will provide chronic pain relief which lasts longer than comparable pain relief that can be provided by either drug alone, and wherein the combination causes lower levels of adverse side effects than either drug administered by itself would cause at a dosage which provides comparable short-term relief from chronic pain.

7. The pharmacological mixture of claim 6 wherein the first drug is selected from the group consisting of memantine, and prodrugs, salts, isomers, analogs and derivatives thereof which are pharmaceutically acceptable, and which suppresses activity at NMDA-type glutamate receptors.

8. The method according to claim 6 wherein the second drug is selected from the group consisting of clonidine, iodoclonidine, guanabenz, guanfacine, xylazine, lofexidine, medetomidine, dexmedetomidine, tizanidine, rilmenidine, azepexole, α-methyldopa, and α-methylnoradrenaline, and salts, isomers, analogs and derivatives thereof which are pharmacologically acceptable, and which stimulate activity at α2 adrenergic receptors.

9. The pharmacological mixture of claim 6 wherein the first drug and the second drug are both present in a unitary dosage form.

10. The pharmacological mixture of claim 9 wherein the unitary dosage form is designed for oral ingestion.

11. The pharmacological mixture of claim 10 wherein the unitary dosage form is selected from the group consisting of tablets and capsules.

12. The pharmacological mixture of claim 9 wherein the unitary dosage form comprises a skin patch.

13. The pharmacological mixture of claim 9, wherein the unitary dosage form is provided by controlled concentrations of the first drug and the second drug in a nondivided material selected from the group consisting of liquids, aerosols, and powders.

14. The pharmacological mixture of claim 13, wherein the nondivided material is packaged in a dispensing device that is capable of dispensing a predetermined volume of the nondivided material each time the device is used.

15. A pharmacological article of manufacture comprising a unitary dosage formulation which contains:
(a) a first drug which is an adamantane derivative which suppresses activity at NMDA-type glutamate receptors; and,
(b) a second drug which stimulates activity at alpha-2 adrenergic receptors,
wherein the first and second drugs are combined with each other in the unitary dosage formulation in dosages which, when combined, provide synergistic and therapeutically effective relief from chronic pain, wherein such relief lasts longer than comparable pain relief provided by either drug alone, and wherein the combination causes lower levels of adverse side effects than either drug administered by itself would cause at a dosage which provides comparable short-term relief from chronic pain.

16. The pharmacological article of manufacture of claim 15 wherein the first drug and the second drug are both present in a unitary dosage form.

17. The pharmacological mixture of claim 16 wherein the unitary dosage form is selected from the group consisting of tablets and capsules.

18. The pharmacological article of manufacture of claim 16 wherein the unitary dosage formulation comprises a skin patch.

19. The pharmacological article of manufacture of claim 16 wherein the unitary dosage form is provided by controlled concentrations of the first drug and the second drug in a nondivided material selected from the group consisting of liquids, aerosols, and powders.

20. The pharmacological mixture of claim 19, wherein the nondivided material is packaged in a dispensing device that is capable of dispensing a predetermined volume of the nondivided material each time the device is used.
